(19) United States
(12) Patent Application Publication
(10) Pub. No.: US 2026/0116839 A1
(43) Pub. Date: Apr. 30, 2026

(54) APPARATUS AND METHOD FOR MANUFACTURING ISOBUTYRIC ACID

(71) Applicant: NAN YA PLASTICS CORPORATION, TAIPEI (TW)

(72) Inventors: CHING-YAO YUAN, TAIPEI (TW); CHUNG-YU CHEN, TAIPEI (TW); YI-CHENG LI, TAIPEI (TW); Tzu-Huan Wong, Hualien County (TW)

(21) Appl. No.: 19/002,619

(22) Filed: Dec. 26, 2024

(30) Foreign Application Priority Data
Oct. 29, 2024 (TW) 113141163

(51) Int. Cl. C07C 51/235; B01J 4/00; B01J 10/00; B01J 19/00; B01J 19/18

(52) U.S. Cl. CPC C07C 51/235; B01J 4/007; B01J 4/008; B01J 10/002; B01J 19/0066; B01J 19/18; B01J 2204/002

(57) ABSTRACT

An apparatus and a method for manufacturing an isobutyric acid. The apparatus includes a reaction tank, air inlet and outlet tubes, and a stirrer in the reaction tank. The air inlet and outlet tubes are connected to the reaction tank, so as to respectively introduce a working gas into the reaction tank and guide a reacted gas out of the reaction tank. The stirrer includes a hollow shaft and blades. An air suction hole is formed on a first end of the hollow shaft and exposed from a liquid surface of the reaction tank. An air discharge hole is formed on a second end of the hollow shaft and immersed into the liquid surface. A condenser and an end of the air outlet tube opposite to the reaction tank are connected for condensation of the reacted gas, so as to obtain isobutyraldehyde and an exhaust gas.

### CROSS-REFERENCE TO RELATED PATENT APPLICATION

This application claims the benefit of priority to Taiwan Patent Application No. 113141163, filed on Oct. 29, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an apparatus and a method for manufacturing an isobutyric acid, and more particularly to an apparatus and a method for manufacturing an isobutyric acid with high efficiency and safety.

### BACKGROUND OF THE DISCLOSURE

An isobutyric acid is a short-chain saturated fatty acid of four carbon atoms. The isobutyric acid is often used for synthesis of isobutyric acid esters, and has an economic value for being applicable in the field of spice, pharmaceutical, and food preservatives. Conventionally, the existing isobutyric acid is manufactured by oxidation of isobutyraldehyde and oxidation of isobutanol. However, byproducts from the oxidation of isobutanol are considerable, thereby resulting in high manufacturing costs and a low yield. Currently, the main method for manufacturing the isobutyric acid is still the oxidation of isobutyraldehyde.

As shown in FIG. 1, a conventional reaction tank R uses a rotation shaft A of a stirrer M to drive rotation of blades L, so as to achieve an effect of stirring and mixing a reaction gas G and a reaction mixture. However, a yield of the conventional reaction tank R is significantly decreased as a result of low contact efficiency between the reaction gas G and the reaction mixture, and this is unbeneficial for industrial manufacturing. The yield can be even lower in response to a low oxygen concentration. Furthermore, due to use of a mechanical seal, leakage and static electricity are likely to occur (which can be dangerous to a reaction process). During the oxidation of isobutyraldehyde, contact efficiency between a gas and a liquid is low, thereby resulting in problems of a poor mixing effect, low reaction selectivity, considerable byproducts, etc.

In the technology of the reaction process, a vapor phase concentration of an organic solvent can be controlled below a lower explosion limit by adjusting operation parameters (decreasing the temperature or increasing the pressure), so as to remove combustibles as a risk factor. In addition, by improving the material of an apparatus, an igniter (static electricity) as a risk factor can be removed. However, since the concentration of oxygen (a combustion-supporting agent) that is used is greater than a limiting oxygen concentration (LOC) for maintaining a high yield, the combustion-supporting agent as a risk factor is still present in the whole reaction process.

Therefore, how to enhance the yield and safety for manufacturing the isobutyric acid through improvements on a manufacturing process, so as to overcome the above-mentioned problems, has become one of the important issues to be solved in the relevant industry.

### SUMMARY OF THE DISCLOSURE

In response to the above-referenced technical inadequacies, the present disclosure provides an apparatus and a method for manufacturing an isobutyric acid.

In order to solve the above-mentioned problems, one of the technical aspects adopted by the present disclosure is to provide an apparatus for manufacturing an isobutyric acid, which includes: a reaction tank, an air inlet tube, an air outlet tube, and a stirrer. The air inlet tube is connected to the reaction tank, so as to introduce a working gas into the reaction tank. The air outlet tube is connected to the reaction tank, so as to guide a reacted gas out of the reaction tank. The stirrer is disposed in the reaction tank, and includes a hollow shaft and a plurality of blades. The hollow shaft has a first end and a second end that are oppositely arranged, an air suction hole is formed on the first end and exposed from a liquid surface of the reaction tank, and an air discharge hole is formed on the second end and immersed into the liquid surface. A condenser and an end of the air outlet tube that is opposite to the reaction tank are connected for condensation of the reacted gas, so as to obtain isobutyraldehyde and an exhaust gas. The isobutyraldehyde is recycled back to the reaction tank by a pump, so as to increase a conversion rate.

In one of the possible or preferred embodiments, the condenser includes a first tube and a second tube, the exhaust gas is discharged from the first tube, and the isobutyraldehyde is discharged from the second tube.

In one of the possible or preferred embodiments, a concentration sensor is further disposed on the first tube.

In one of the possible or preferred embodiments, the air outlet tube further includes a manometer and a control valve.

In one of the possible or preferred embodiments, the working gas is air, nitrogen, or oxygen.

In order to solve the above-mentioned problems, another one of the technical aspects adopted by the present disclosure is to provide a method for manufacturing an isobutyric acid, which uses the apparatus mentioned above. The method includes: supply of the isobutyraldehyde, in which the isobutyraldehyde is injected into the reaction tank; atmosphere replacement, in which the atmosphere replacement is performed by injecting nitrogen into the reaction tank through the air inlet tube; and an oxidation reaction, in which the working gas is injected into the reaction tank through the air inlet tube, and the isobutyraldehyde is enabled to react with the working gas until a reaction end point is reached, so as to obtain the isobutyric acid.

In one of the possible or preferred embodiments, the atmosphere replacement further includes using a control valve to control a pressure between 1 kg/cm² and 10 kg/cm².

In one of the possible or preferred embodiments, the atmosphere replacement is completed when an oxygen content of the exhaust gas is less than 0.005%.

In one of the possible or preferred embodiments, in the oxidation reaction, an oxygen content of the exhaust gas ranges between 0.005% and 8%.

In one of the possible or preferred embodiments, the reaction end point is reached when an oxygen content of the exhaust gas ranges between 7% and 8%.

Therefore, in the apparatus and the method for manufacturing the isobutyric acid provided by the present disclosure, by virtue of “the stirrer including a hollow shaft and a plurality of blades, the hollow shaft having a first end and a second end that are oppositely arranged, an air suction hole being formed on the first end and exposed from a liquid surface of the reaction tank, and an air discharge hole being formed on the second end and immersed into the liquid surface” and “a condenser and an end of the air outlet tube that is opposite to the reaction tank being connected for condensation of the reacted gas, so as to obtain isobutyraldehyde and an exhaust gas,” the isobutyraldehyde can be recycled back to the reaction tank by the pump, so as to increase the conversion rate. In addition, a yield and safety for manufacturing the isobutyric acid can be enhanced.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic diagram of a conventional reaction tank in the related art;

FIG. 2 is a schematic diagram of an apparatus for manufacturing an isobutyric acid according to the present disclosure;

FIG. 3 is a schematic side view of a stirrer of the apparatus for manufacturing the isobutyric acid according to the present disclosure;

FIG. 4 is a schematic top view of the stirrer of the apparatus for manufacturing the isobutyric acid according to the present disclosure;

FIG. 5 is a flowchart of a method for manufacturing the isobutyric acid according to the present disclosure; and

FIG. 6 is a schematic diagram showing changes in oxygen concentration during a reaction process in the method for manufacturing the isobutyric acid according to the present disclosure.

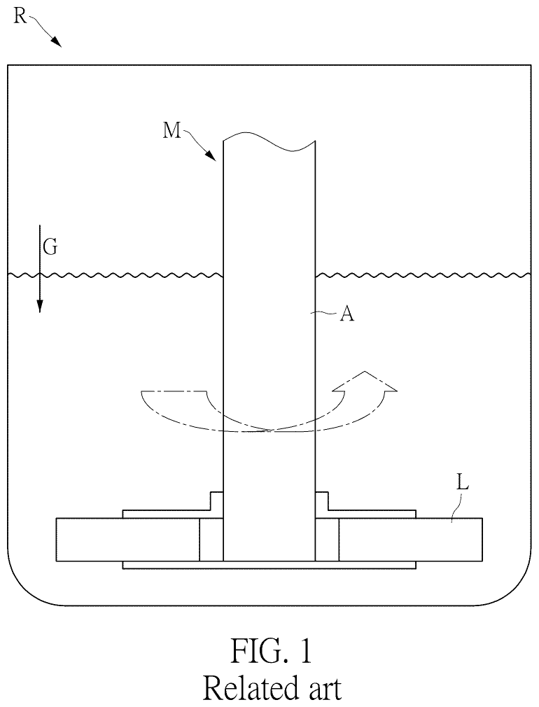

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

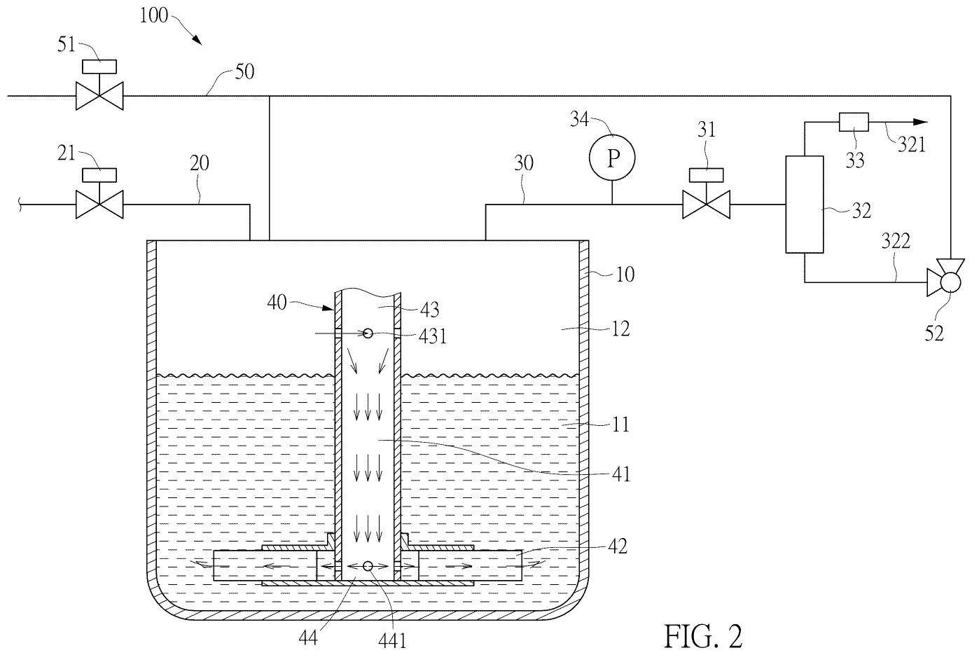

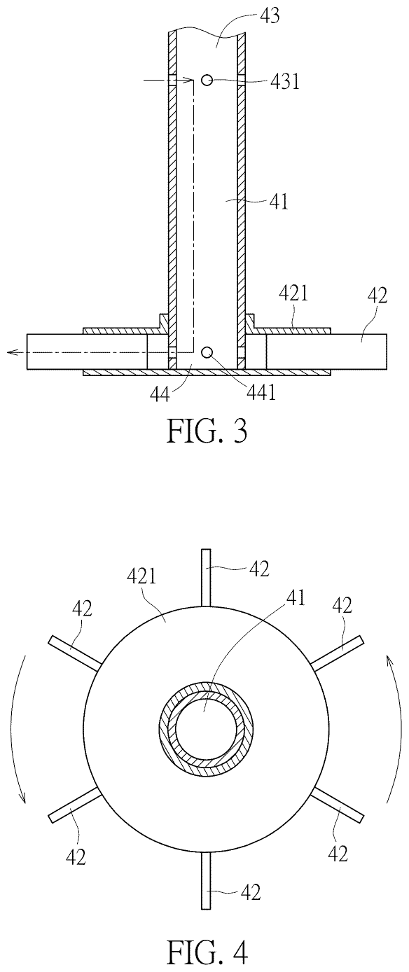

Referring to FIG. 2 to FIG. 4, a first embodiment of the present disclosure provides an apparatus 100 for manufacturing an isobutyric acid. The apparatus 100 includes a reaction tank 10, an air inlet tube 20, an air outlet tube 30, and a stirrer 40. The reaction tank 10 is a sealed tank that can accommodate a reaction mixture 11. In the present disclosure, the reaction mixture 11 is isobutyraldehyde. Specifically, the isobutyraldehyde flows into the reaction tank 10 through an isobutyraldehyde feed tube 50. In one embodiment, a third control valve 51 is disposed on the isobutyraldehyde feed tube 50, so as to control an amount of the isobutyraldehyde that enters the reaction tank 10. The air inlet tube 20 is connected to the reaction tank 10, so as to introduce a working gas 12 into the reaction tank 10. In the present disclosure, the working gas 12 can be air, nitrogen, or oxygen.

Generally, a gas present in the reaction tank 10 is a common atmosphere (i.e., air). Before an oxidation reaction, nitrogen is introduced into the reaction tank 10 through the air inlet tube 20, so that an atmospheric pressure in the reaction tank 10 is maintained between 1 kg/cm² and 10 kg/cm². Afterwards, oxygen is introduced as an oxidant, and the oxidation reaction occurs between the oxygen and the isobutyraldehyde. In another embodiment of the present disclosure, the air can also be introduced as the oxidant. Since the air is easily obtainable and separable with the isobutyric acid, a manufacturing process can be simplified, manufacturing costs can be reduced, and safety can be enhanced.

Specifically, in the present disclosure, the stirrer 40 of the apparatus 100 includes a hollow shaft 41 and a plurality of blades 42 that are fixed onto an impeller 421. The hollow shaft 41 has a first end 43 and a second end 44 that are oppositely arranged. An air suction hole 431 is formed on the first end 43 and exposed from a liquid surface of the reaction tank 10. An air discharge hole 441 is formed on the second end 44 and immersed into the liquid surface. Hence, when being introduced, the oxygen or the air can be guided from the air suction hole 431 at the first end 43 to the air discharge hole 441 at the second end 44. By guiding the oxygen or the air to a bottom portion of the reaction mixture 11 in the reaction tank 10, mixing efficiency of the gas and a liquid can be improved. That is to say, the stirrer 40 of the present disclosure has functions of air suction, air discharge, and stirring.

In one embodiment of the present disclosure, a first control valve 21 is disposed on the air inlet tube 20, and a second control valve 31 is disposed on the air outlet tube 30. In this way, turning on and turning off of the air inlet tube 20 and the air outlet tube 30 can be controlled in a respective manner, so as to further control the oxidation reaction carried out in the reaction tank 10. For example, the first control valve 21 and the second control valve 31 are simultaneously turned on, so that the working gas 12 is introduced into the reaction tank 10 for replacement of the gas in the reaction tank 10. Then, the second control valve 31 is turned off, and the working gas 12 is continuously introduced from the air inlet tube 20 to perform pressurization. The oxygen or the air is further introduced to carry out the oxidation reaction. In the present disclosure, each of the first control valve 21 and the second control valve 31 can be a back pressure valve. However, the aforementioned examples describe only one of the embodiments of the present disclosure, and the present disclosure is not intended to be limited thereto. In one embodiment of the present disclosure, the gas in the reaction tank 10 undergoes the oxidation reaction when the nitrogen and the oxygen are at a ratio of 99.995:0.005, so as to achieve preferable reaction efficiency.

The air outlet tube 30 is connected to the reaction tank 10, so as to guide a reacted gas out of the reaction tank 10. In addition, a condenser 32 and an end of the air outlet tube 30 that is opposite to the reaction tank 10 are connected for condensation of the reacted gas, so as to obtain the isobutyraldehyde and an exhaust gas. The condensed isobutyraldehyde can be re-guided to the reaction tank 10, so that a conversion rate is increased. Specifically, the condenser 32 includes a first tube 321 and a second tube 322, the first tube 321 is connected to an upper end of the condenser 32 to discharge the exhaust gas, and the second tube 322 is connected to a lower end of the condenser 32 to discharge the isobutyraldehyde. In one embodiment of the present disclosure, a manometer 34 is further disposed on the air outlet tube 30, so as to monitor a pressure in the reaction tank 10.

In one embodiment of the present disclosure, a concentration sensor 33 is further disposed on the first tube 321, so as to monitor a concentration of the exhaust gas. Specifically, the exhaust gas can be the oxygen, the air, and the nitrogen. By using the concentration sensor 33 to monitor an oxygen concentration and an isobutyraldehyde concentration at an exit end, a reacted oxygen concentration is ensured to be lower than a limiting oxygen concentration (LOC). Furthermore, a reaction end point is controlled, so that reaction performance and safety of the oxidation reaction can be maintained.

### Second Embodiment

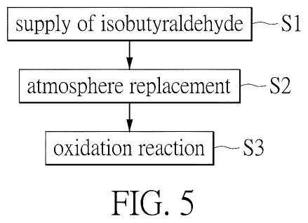

Referring to FIG. 5, a second embodiment of the present disclosure provides a method for manufacturing an isobutyric acid, and the method uses the apparatus mentioned above. The method at least includes the following steps: supply of isobutyraldehyde (step S1), in which the isobutyraldehyde is injected into the reaction tank 10; atmosphere replacement (step S2), in which the atmosphere replacement is performed by injecting nitrogen into the reaction tank 10 through the air inlet tube 20; and an oxidation reaction (step S3), in which the working gas 12 is injected into the reaction tank 10 through the air inlet tube 20, and the isobutyraldehyde is enabled to react with the working gas 12 until a reaction end point is reached, so as to obtain the isobutyric acid.

In step S1, the isobutyraldehyde is placed into the reaction tank 10, and is stirred by the stirrer 40. In the present disclosure, the stirrer 40 is a hollow stirrer, and has the air suction hole 431 formed on the first end 43 and the air discharge hole 441 formed on the second end 44, thereby allowing a gas to flow in the hollow shaft 41. In this way, oxygen or air can be guided to a bottom portion of the reaction mixture 11 in the reaction tank 10, so as to enhance mixing efficiency of the gas and a liquid.

In the atmosphere replacement (step S2), the second control valve 31 is used to control a pressure between 1 kg/cm² and 10 kg/cm² (e.g., any positive real number between 1 and 10). In one embodiment of the present disclosure, a flow velocity of the working gas 12 that is guided by the air inlet tube 20 can range between 1 L/min and 10 L/min (e.g., any positive real number between 1 and 10). Then, an oxygen content of an exhaust gas is monitored by the concentration sensor 33, and the atmosphere replacement is completed when the oxygen content of the exhaust gas is less than 0.005%.

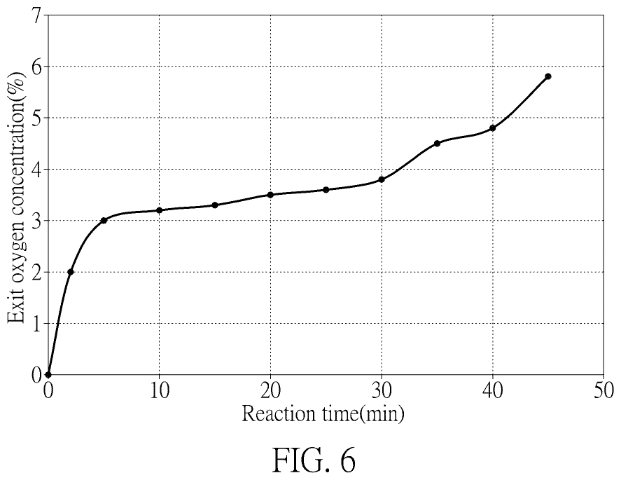

In the oxidation reaction (step S3), a reaction temperature can range between 20° C. and 100° C., and a reaction pressure can range between 1 kg/cm² and 10 kg/cm². After the reaction, the isobutyric acid will remain in the reaction tank 10 due to its high boiling point and relative non-volatility, and the isobutyraldehyde will be discharged out of the reaction tank 10 through the air outlet tube 30. In the present disclosure, a conversion rate of the isobutyraldehyde is at least 90%. It should be noted that the oxygen content of the exhaust gas during the oxidation reaction ranges between 0.005% and 8% (e.g., any positive real number between 0.005 and 8), so as to prevent an oxygen concentration from falling within an explosion range. Finally, when the oxygen content of the exhaust gas ranges between 7% and 8% (e.g., 7.2%, 7.4%, 7.6%, or 7.8%), the reaction end point is reached. At this time, the liquid in the reaction tank 10 is the isobutyric acid. Referring to Table 1 below, FIG. 6 is a schematic diagram showing changes in oxygen concentration during a reaction process in the method for manufacturing the isobutyric acid according to the present disclosure. When the oxygen content of the exhaust gas ranges between 7% and 8%, it is indicated that the input oxygen will no longer be consumed and the reaction end point is reached.

TABLE 1

| | Exit Oxygen |
|---|---|
| Reaction Time (min) | Concentration (%) |
| 0 | 0 |
| 2 | 2 |
| 5 | 3 |
| 10 | 3.2 |
| 15 | 3.3 |
| 20 | 3.5 |
| 25 | 3.6 |
| 30 | 3.8 |
| 35 | 4.5 |
| 40 | 4.8 |
| 45 | 5.8 |
| 50 | 6.7 |
| 55 | 7.8 |
| 60 | 8 |

A fourth control valve 52 can be turned on or off to control an amount of the isobutyraldehyde that is discharged from the second tube 322 and flows into the reaction tank 10 after flowing back to the isobutyraldehyde feed tube 50. The overall conversion rate of the isobutyraldehyde can be further increased by recycling and reusing the reacted isobutyraldehyde. In one embodiment of the present disclosure, the fourth control valve 52 can be a ball valve, so as to be quickly turned on or off.

As shown in Table 1 above, the oxygen concentration will be increased in response to an increase in time. As shown in Table 2 below, a limiting oxygen concentration of the isobutyraldehyde is 8.8%, and a limiting oxygen concentration of the isobutyric acid is 8%. As such, a value of an exit oxygen concentration can be set in the method of the present disclosure, and the reaction is stopped when said value is reached, so as to avoid the risk of explosion.

TABLE 2

| | | | Limiting Oxygen |
|---|---|---|---|
| | Lower Limit of | Upper Limit of | Concentration |
| Chemicals | Explosion (%) | Explosion (%) | (LOC) |
| Isobutyraldehyde | 1.6% | 10.6% | 8.8% |
| Isobutyric acid | 1.6% | 7.3% | 8% |

Examples of the present disclosure are as shown in Table 3 below. In Table 3, a product in a reaction tank is taken and subjected to gas chromatography (GC) analysis. Here, the conversion rate is a number of moles of an isobutyraldehyde residue/a number of moles of an isobutyraldehyde feed*100%, and a selectivity is a number of moles of the isobutyric acid/the number of moles of the isobutyraldehyde feed*100%.

TABLE 3

| | | Reaction | | | Conversion | Selectivity |
|---|---|---|---|---|---|---|
| | Type of | Temper- | Reaction | | Rate (%) of | (%) of |
| | Reaction | ature | Time | Pressure | Isobutyr- | Isobutyric |
| Example | Tank | (° C.) | (min) | (kg/cm²) | aldehyde | Acid |
| 1 | common | 30 | 60 | 1 | 40 | 81 |
| | stirred tank | | | | | |
| 2 | hollow-shaft | 30 | 60 | 1 | 70 | 85 |
| | stirred tank | | | | | |
| 3 | hollow-shaft | 60 | 60 | 1 | 73 | 86 |
| | stirred tank | | | | | |
| 4 | hollow-shaft | 60 | 60 | 5 | 85 | 91 |
| | stirred tank | | | | | |
| 5 | hollow-shaft | 80 | 60 | 5 | 86 | 90 |
| | stirred tank | | | | | |
| 6 | hollow-shaft | 60 | 60 | 10 | 87.5 | 90 |
| | stirred tank | | | | | |
| 7 | hollow-shaft | 80 | 60 | 10 | 90 | 93 |
| | stirred tank | | | | | |

From Table 3 above, it can be observed that the difference between Example 1 and Example 2 is the type of the reaction tank. With the same reaction temperature, reaction time, and pressure, the mixing efficiency of the gas and the liquid can be enhanced by use of a hollow-shaft stirred tank, thereby resulting in an enhanced selectivity of the isobutyric acid and a significantly increased conversion rate of the isobutyraldehyde. The difference between Example 2 and Example 3 is a temperature rise. A comparison between the results of Example 2 and Example 3 shows that the selectivity of the isobutyric acid and the conversion rate of the isobutyraldehyde can be further improved when the reaction temperature is increased from 30° C. to 60° C. The difference between Example 3 and Example 4 is a pressure increase. A comparison between the results of Example 3 and Example 4 shows that the selectivity of the isobutyric acid and the conversion rate of the isobutyraldehyde can be further improved when the pressure is increased from 1 kg/cm² to 5 kg/cm².

Furthermore, the difference between Example 4 and Example 5 is a temperature rise. A comparison between the results of Example 4 and Example 5 shows that the selectivity of the isobutyric acid and the conversion rate of the isobutyraldehyde can be further improved when the reaction temperature is increased from 60° C. to 80° C. The difference between Example 5 and Example 6 is a pressure increase (while there is a decrease in the reaction temperature). A comparison between the results of Example 5 and Example 6 shows that, when the pressure is increased from 5 kg/cm² to 10 kg/cm² (even though the reaction temperature is decreased from 80° C. to 60° C.), the selectivity of the isobutyric acid and the conversion rate of the isobutyraldehyde can still be improved. In an exemplary example of the present disclosure, the selectivity of the isobutyric acid can reach 93% and the conversion rate of the isobutyraldehyde can reach 90% when the reaction is carried out for 60 minutes at a reaction temperature of 80° C. and a pressure of 10 kg/cm².

### Beneficial Effects of the Embodiments

In conclusion, in the apparatus and the method for manufacturing the isobutyric acid provided by the present disclosure, by virtue of “the stirrer including a hollow shaft and a plurality of blades, the hollow shaft having a first end and a second end that are oppositely arranged, an air suction hole being formed on the first end and exposed from a liquid surface of the reaction tank, and an air discharge hole being formed on the second end and immersed into the liquid surface” and “a condenser and an end of the air outlet tube that is opposite to the reaction tank being connected for condensation of the reacted gas, so as to obtain isobutyraldehyde and an exhaust gas,” a yield and safety for manufacturing the isobutyric acid can be enhanced.

Specifically, the apparatus of the present disclosure is a magnetic seal stirrer that can guide a gas by having functions of air suction, air discharge, and stirring, so that a gas-liquid transmission rate can be effectively increased. When an oxidation reaction is carried out at a low oxygen concentration, a reaction yield can be further improved. Since a magnetic (dry-running) seal is used in the apparatus of the present disclosure, leakage and static electricity can be prevented to enhance safety of a reaction process. Moreover, in the apparatus of the present disclosure, a concentration sensor is disposed on a first tube, so as to monitor a concentration of an exhaust gas, detect an oxygen concentration at an exit end of a reactor, control an exit isobutyraldehyde concentration and a reaction end point, and improve the safety.

Furthermore, since the apparatus of the present disclosure includes a hollow stirrer, efficiency of the oxidation reaction can be enhanced without addition of a catalyst, thereby saving the costs for manufacturing the isobutyric acid.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An apparatus for manufacturing an isobutyric acid, comprising:
a reaction tank;
an air inlet tube, wherein the air inlet tube is connected to the reaction tank, so as to introduce a working gas into the reaction tank;
an air outlet tube, wherein the air outlet tube is connected to the reaction tank, so as to guide a reacted gas out of the reaction tank; and
a stirrer disposed in the reaction tank, wherein the stirrer includes a hollow shaft and a plurality of blades, the hollow shaft has a first end and a second end that are oppositely arranged, an air suction hole is formed on the first end and exposed from a liquid surface of the reaction tank, and an air discharge hole is formed on the second end and immersed into the liquid surface;
wherein a condenser and an end of the air outlet tube that is opposite to the reaction tank are connected for condensation of the reacted gas, so as to obtain isobutyraldehyde and an exhaust gas.

2. The apparatus according to claim 1, wherein the condenser includes a first tube and a second tube, the exhaust gas is discharged from the first tube, and the isobutyraldehyde passes through the second tube and is returned to the reaction tank for further reaction.

3. The apparatus according to claim 2, wherein a concentration sensor is further disposed on the first tube; wherein, when an oxygen content of the exhaust gas ranges between 7% and 8%, a residual liquid in the reaction tank is the isobutyric acid.

4. The apparatus according to claim 1, wherein the air outlet tube further includes a manometer and a control valve.

5. The apparatus according to claim 1, wherein the working gas is air, nitrogen, or oxygen.

6. A method for manufacturing an isobutyric acid, which uses the apparatus as claimed in claim 1, the method comprising:
supply of the isobutyraldehyde, wherein the isobutyraldehyde is injected into the reaction tank;
atmosphere replacement, wherein the atmosphere replacement is performed by injecting nitrogen into the reaction tank through the air inlet tube; and
an oxidation reaction, wherein the working gas is injected into the reaction tank through the air inlet tube, and the isobutyraldehyde is enabled to react with the working gas until a reaction end point is reached, so as to obtain the isobutyric acid.

7. The method according to claim 6, wherein the atmosphere replacement further includes using a control valve to control a pressure between 1 kg/cm² and 10 kg/cm².

8. The method according to claim 6, wherein the atmosphere replacement is completed when an oxygen content of the exhaust gas is less than 0.005%.

9. The method according to claim 6, wherein, in the oxidation reaction, an oxygen content of the exhaust gas ranges between 0.005% and 8%.

10. The method according to claim 6, wherein the reaction end point is reached when an oxygen content of the exhaust gas ranges between 7% and 8%.
